(19) United States
(12) Patent Application Publication
(10) Pub. No.: US 2026/0284317 A1
(43) Pub. Date: Sep. 24, 2026

(54) MIXING SYRINGE ASSEMBLIES AND METHODS

(71) Applicant: CLEARSTREAM TECHNOLOGIES LIMITED, Enniscorthy, County Wexford (IE)

(72) Inventor: Matthew Cullen, Wicklow Town (IE)

(73) Assignee: CLEARSTREAM TECHNOLOGIES LIMITED, Enniscorthy, County Wexford (IE)

(21) Appl. No.: 19/471,877

(22) PCT Filed: Apr. 26, 2024
(86) PCT No.: PCT/US24/26519

§ 371 Date: Oct. 2, 2025

Related U.S. Application Data
(60) Provisional application No. 63/498,676, filed on Apr. 27, 2023.

(51) Int. Cl. A61M 5/28; A61M 5/19

(52) U.S. Cl. CPC A61M 5/284; A61M 5/19; A61M 2202/04

(57) ABSTRACT

A mixing syringe assembly for mixing two constituent materials includes a barrel body, and a barrel connector. The barrel body has a first portion and a second portion. The barrel connector connects the first portion to the second portion includes a twisting seal having a plurality of flaps. The twisting seal is operable to be actuated from a closed configuration wherein the twisting seal seals the first portion from the second portion and an open configuration in which the plurality of flaps twist apart to provide an opening between the first portion and the second portion.

### CROSS-REFERENCE TO RELATED APPLICATIONS

The present application claims the benefit of U.S. Provisional Application No. 63/498,676, entitled “MIXING SYRINGE ASSEMBLIES AND METHODS OF MIXING CONSTITUENT MATERIALS,” the entirety of which is incorporated by reference herein.

### BACKGROUND

### Field

The present disclosure generally relates to mixing syringe assemblies and methods and, more particularly, mixing syringe assemblies for delivering radiation spacer material and associated methods.

### Technical Background

Prostate cancer is the most common non-skin cancer diagnosed in men. Radiation therapy is an excellent treatment option for prostate cancer. However, radiation exposure can cause unintended side effects in adjacent non-targeted tissue. A radiation spacer, such as a radio protective spacer) can be implanted to avoid collateral radiation and minimize injury to nearby tissues by providing a space between the target tissue and non-targeted tissues at risk.

Conventional radiation spacers may include gelling materials, which are delivered as a liquid and allowed to cure. However, constituent components of the gel may begin to gel on contact with one another, which may make delivery difficult. Moreover, constituent components may need to remain separate until time of use, otherwise they may degrade over time due to limited stability.

### SUMMARY

Embodiments of the present disclosure are directed to various mixing syringe assemblies, which may maintain separation of constituent materials until time for use of the mixed constituent materials.

In one embodiment, a mixing syringe assembly for mixing two constituent materials includes a barrel body having a first portion and a second portion, and a barrel connector connecting the first portion to the second portion. The barrel connector includes a twisting seal having a plurality of flaps, wherein the twisting seal is operable to be actuated from a closed configuration wherein the twisting seal seals the first portion from the second portion and an open configuration in which the plurality of flaps twist apart to provide an opening between the first portion and the second portion.

In another embodiment, pre-loaded mixing syringe assembly includes a barrel body having a first portion and a second portion, a barrel connector connecting the first portion to the second portion, a first constituent material housed in the first portion, and a second constituent material housed in the second portion. The barrel connector has a twisting seal having a plurality of flaps, wherein the twisting seal is operable to be actuated from a closed configuration wherein the twisting seal seals the first portion from the second portion and an open configuration in which the plurality of flaps twist apart to provide an opening between the first portion and the second portion. In the closed configuration the twisting seal prevents the first constituent material from mixing with the second constituent material and in the open configuration the first constituent material is mixable with the second constituent material.

In yet another embodiment, a method of assembling a mixing syringe assembly is disclosed. The method includes positioning a first constituent material in a first portion of a barrel body, positioning a second constituent in a second portion of the barrel body, and connecting the first portion of the barrel body to the second portion of the barrel body with a barrel connector. The barrel connector includes a twisting seal having a plurality of flaps, wherein the twisting seal is operable to be actuated from a closed configuration wherein the twisting seal seals the first portion from the second portion and an open configuration in which the plurality of flaps twist apart to provide an opening between the first portion and the second portion.

Additional features and advantages of the aspects described herein will be set forth in the detailed description, which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 schematically depicts an embodiment of a syringe assembly for mixing constituent materials, according to one or more embodiments shown and described herein;

FIG. 2 schematically depicts a cross-section of a barrel connector of the syringe assembly of FIG. 1, according to one or more embodiments shown and described herein;

FIG. 3A schematically depicts a barrel connector having a tri-flap seal, according to one or more embodiments shown and described herein;

FIG. 3B schematically depicts a barrel connector having an overlapping tri-flap seal, according to one or more embodiments shown and described herein;

FIG. 3C schematically depicts a barrel connector having a twisting seal, according to one or more embodiments shown and described herein;

FIG. 3D schematically depicts a cross-section of the barrel connector of FIG. 3C in a closed configuration, according to one or more embodiments shown and described herein;

FIG. 3E schematically depicts the twisting seal of FIG. 3C actuated to an open configuration, according to one or more embodiments shown and described herein;

FIG. 4 schematically depicts a syringe assembly for mixing constituent materials, according to one or more embodiments shown and described herein;

FIG. 5 schematically depicts a cross-section of a barrel connector, according to one or more embodiments shown and described herein;

FIG. 6 schematically depicts a dual barrel syringe assembly, according to one or more embodiments shown and described herein;

FIG. 7 schematically depicts a barrel body having dual barrels in isolation from the dual barrel assembly of FIG. 6, according to one or more embodiments shown and described herein;

FIG. 8 schematically depicts a dual barrel mixing syringe assembly, according to one or more embodiments shown and described herein; and

FIG. 9 depicts a flow chart illustrating a method of assembling a mixing syringe assembly, according to one or more embodiments shown and described herein.

### DETAILED DESCRIPTION

The present disclosure is generally directed to mixing syringe assemblies for mixing at least two constituent materials. Particularly, the present application is directed to mixing syringe assemblies configured for delivering a radiation spacer. For example, radiation spacers may be formed of one or more hydrogel materials, which may be delivered to a desired location, such as within a balloon or on its own and cured in place to block or substantial block radiation, which may be unintentionally directed toward healthy tissue instead of targeted/diseased tissue. During mixing of the hydrogel for forming a radiation spacer, multiple materials may need to be combined just prior to and/or at delivery of the radiation spacer into a target location within the body. To maintain quality of material, powder components may need to be kept dry or un-constituted prior to application. However, during use, powder components may need to be hydrated or mixed with a diluent. It may be difficult to determine proper mixing volumes, maintain sterility, and prevent unwanted early mixing using traditional methods. However, embodiments of the present disclosure may provide constituent materials in prepackaged, pre-measured, ready-to use syringe assemblies, which, as will be described in greater detail herein, provide improved mixing, maintains sterility, and/or improves delivery.

Particular embodiments of a mixing syringe assembly include a barrel body having a first portion and a second portion and a barrel connector connecting the first portion to the second portion. The barrel connector is also operable as a sealing device to prevent interactions of materials stored in the first portion and the second portion until desired. For example, the barrel connector includes a seal operable to be actuated from a closed configuration to an open configuration when desired to allow mixing of materials. These and additional embodiments and benefits will be described in greater detail below.

Directional terms as used herein-for example up, down, right, left, front, back, top, bottom—are made only with reference to the figures as drawn and are not intended to imply absolute orientation unless otherwise specified.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any device or assembly claim does not actually recite an order or orientation to individual components, or it is not otherwise specifically stated in the claims or description that the steps are to be limited to a specific order, or that a specific order or orientation to components of an device or assembly is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation; and the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

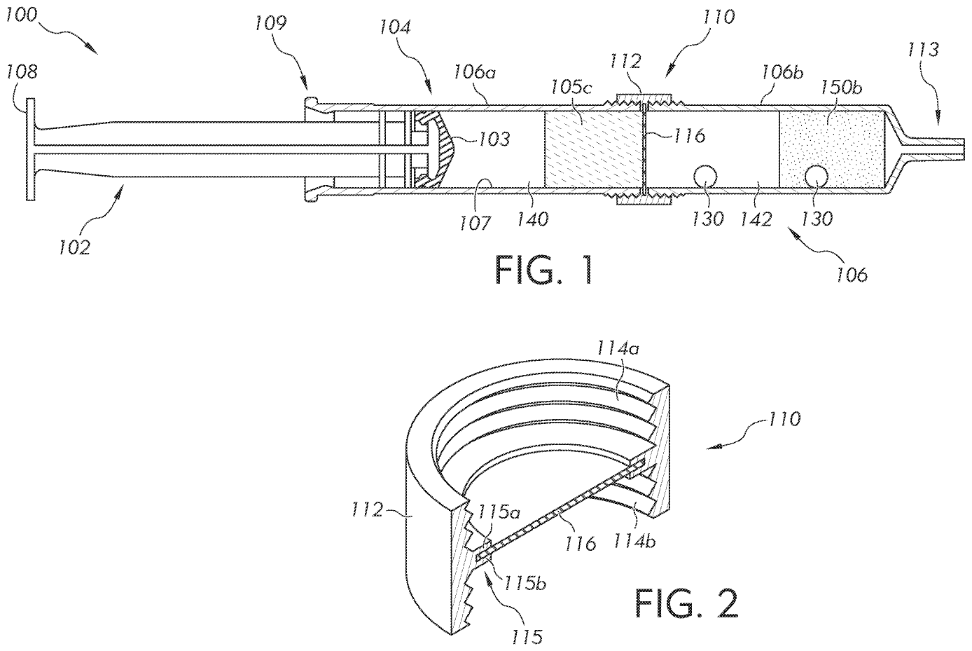

Turning now to the drawings, FIG. 1 depicts an illustrative mixing syringe assembly 100. The mixing syringe assembly 100 may be used for combining two or more constituent materials as will be described in greater detail herein. In particular, the mixing syringe assembly 100 may be used to mix or combine two or more constituent components of a hydrogel for use as a radiation spacer. The mixing syringe assembly 100, as depicted, generally includes a plunger 102, a barrel body 106, and a barrel connector 110. In some embodiments, the mixing syringe assembly 100 further includes one or more mixing masses 130. It is noted that a mixing syringe assembly 100 may include a greater or fewer number of components without departing from the scope of the present disclosure.

The plunger 102 may be slidably disposed in a lumen 107 of the barrel body 106 and may form a fluidic seal with the barrel body 106 via a seal 103 at a distal end of the plunger 102. For example, the seal 103 may be a rubber or similar conformable material for forming a fluid-tight seal with the barrel body 106. At a proximal end of the plunger 102 may be a pusher flange 108 for a user to engage for advancing the plunger 102 along the barrel body 106.

The barrel body 106 may be generally hollow and have a plunger-receiving end 109 at a proximal end for receiving the plunger 102 and a fluid delivery opening 113 (also referred to as a dispensing end) at a distal end, which may be coupled to a material delivery cannula (not shown, which may include a manifold, delivery needle or the like). In embodiments, the fluid delivery opening 113 may be axially positioned with a centerline of the barrel body 106 and/or plunger 102. However, in other embodiments, the fluid delivery opening 113 may be offset from a centerline of the barrel body 106 and/or plunger 102. Such offset may be useful in preventing the one or more mixing masses 130 from blocking the fluid delivery opening 113. The barrel body may be any size barrel such as a 5 ml barrel, 10 ml barrel, etc.

In embodiments, the barrel body 106 is divided into a first portion 106a and a second portion 106b. The first portion 106a and the second portion 106b are connected to one another via the barrel connector 110. As depicted, the first portion 106a and the second portion 106b may have a congruent inner diameter. This may allow the same plunger 102 to pass through both the first portion 106a and the second portion 106b while remaining sealed to the inner wall of both the first portion 106a and the second portion 106b.

The first portion 106a provides a first constituent material section 140 and the second portion 106b provides a second constituent material section 142, wherein separation between the first constituent material section 140 and the second constituent material section 142 is provided by the barrel connector 110. The first constituent material section 140 and the second constituent material section 142 may take up the same volume or substantially same volume as depicted. However, in some embodiments, initial volumes may differ from one another. Embodiments, provided herein may provide a modular assembly, wherein portions 106a, 106b having different volume capacities but equal internal diameters may be connected as desired based on needed volumes for holding constituent materials. As will be described in greater detail, a first constituent material 150a may be positioned initially within the first constituent material section 140 and a second constituent material 150b, different from the first constituent material 150a, may be positioned within the second constituent material section 142. As depicted in the illustrated embodiment, the first constituent material section 140 may be positioned proximal to the second constituent material section 142. Stated another way, the first portion 106a may be positioned proximal to the second portion 106b, which includes the dispensing end 113.

The first constituent material 150a may be a liquid for hydrating the second constituent material 150b, which may be a powder or particulate. For example, the first constituent material 150a may be saline, water, deionized water, or the like. As noted above, the second constituent material 150b may be a powder or particulate material such as but not limited to albumin, polyethylenimine (PEI), an amine containing polyethylene glycol (PEG) or protein, an N-hydroxysuccinimide (NHS) ester component such as PEG-(SS)2, PEG-(SS)4, PEG-(SS)8, PEG-(SG)4, PEG-(SG)8, and/or the like. In some aspects, molecular weights of the PEG components may range from about 2,000 to about 100,000. The powder or particular material may be biodegradable and/or bioabsorbable. As used herein, “biodegradable” and/or “bioabsorbable” refers to a compound that can be absorbed by the surrounding or local tissue of a subject and/or degraded and absorbed by the tissue of the subject.

The powder or particulate material can be composed of various crosslinking substances of varying amounts, designed to allow the hydrogel to last a specific amount of time in situ before degrading. In aspects, the hydrogel components may be selected based on a degradation time that corresponds to the length of anticipated radiation therapy. In aspects the length of anticipated radiation therapy, and thus the targeted time for hydrogel degradation is up to 18 months, for example from the range of about 0 months to about 18 months, including about 1 month, 2 months, 3 months, 4 months, 5 months, 6 months, 7 months, 8 months, 9 months, 10 months, 11 months, 12 months, 13 months, 14 months, 15 months, 16 months, 17 months, and 18 months. It should be understood that the time is merely a rough guide generally used to target appropriate formulation of the hydrogel.

The barrel connector 110 may take on several different forms. In general though, the various barrel connectors 110 described herein may be actuated to selectively provide a flow path between the first constituent material section 140 and the second constituent material section 142 to allow for the first constituent material 150a from the first constituent material section 140 to move into the second constituent material section 142 to allow for mixing of the first constituent material 150a with the second constituent material 150b.

Still referring to FIG. 1, the barrel connector 110 includes a connector body 112 and an inner seal 116. The connector body may be any suitable material (e.g., plastic) and may be operable to connect the first portion 106a to the second portion 106b. In embodiments, the connector body 112 may be internally threaded, as depicted, for coupling to the first portion 106a and the second portion 106b. Accordingly, the first portion 106a and the second portion 106b may externally threaded at least along portions adjacent to one. The connector body 112 may be threaded onto both the first portion 106a and the second portion 106b causing the first portion 106a and the second portion 106b to move axially toward one another. In some embodiments, the inner seal 116 may become sandwiched or compressed between the first portion and the second portion when assembled to one another with the connector body 112. For example, in some embodiments, the inner seal 116 may not be coupled to the connector body 112 but may be held within the connector body 112 by the first portion 106a and the second portion 106b.

The one or more mixing masses 130 may initially be positioned within either the first constituent material section 140 or the second constituent material section 142. It is noted that in embodiments, placing the one or more mixing masses 130 within the second constituent material section 142 may be beneficial in that a flow path large enough to allow the one or more mixing masses 130 through need not be created to move the one or more mixing masses 130 from the first constituent material section 140 to the second constituent material section 142. The one or more mixing masses 130 may be any suitable material to assist in mixing materials such as stainless steel, delron, plastic polycarbonate, composite, aluminum, low carbon steel, chemical-resistant PTFE, composite, titanium, moisture resistant polyethylene, magnetic material, etc. In one or more mixing masses 130, may be any shape such as spherical, cylindrical, cubic, ellipsoid, or any other regular or irregular shape. The one or more mixing masses 130 may include any number of masses such as two or more, three or more, four or more etc. The one or more mixing masses 130 may be identical to one another or different from one another. The one or more mixing masses 130 may have various sizes such as up to 7 mm in diameter, such as up to 6 mm in diameter, such as up to 5 mm in diameter, such as up to 4 mm in diameter, such as up to 3 mm in diameter, such as 7 mm or less in diameter. In various embodiments, the one or more mixing masses 130 may have designs cut or etched into its surface, which may enhance mixing.

Referring to FIG. 2 the barrel connector 110 is schematically depicted in isolation and in cross-section. In some embodiments, the connector body 112 may include a first threaded portion 114a to threadingly engaging the first portion 106a of the barrel body 106 and a second threaded portion 114b to threadingly engage the second portion 106b of the barrel body 106. Between the first threaded portion 114a and the second threaded portion 114b may be a seal holder 115. For example, the seal holder 115 may include radially inwardly extending arms 115a, 115b, which hold the inner seal 116 therebetween. As noted above, in some embodiments, the inner seal 116 may not be coupled or held directly by the connector body 112 but may be held in place by the first portion 106a, the second portion 106b, or both. In some instances, it is contemplated that the inner seal 116 may be first affixed (e.g., via adhesive, welding, or the like) to one of the first portion 106a or the second portion 106b.

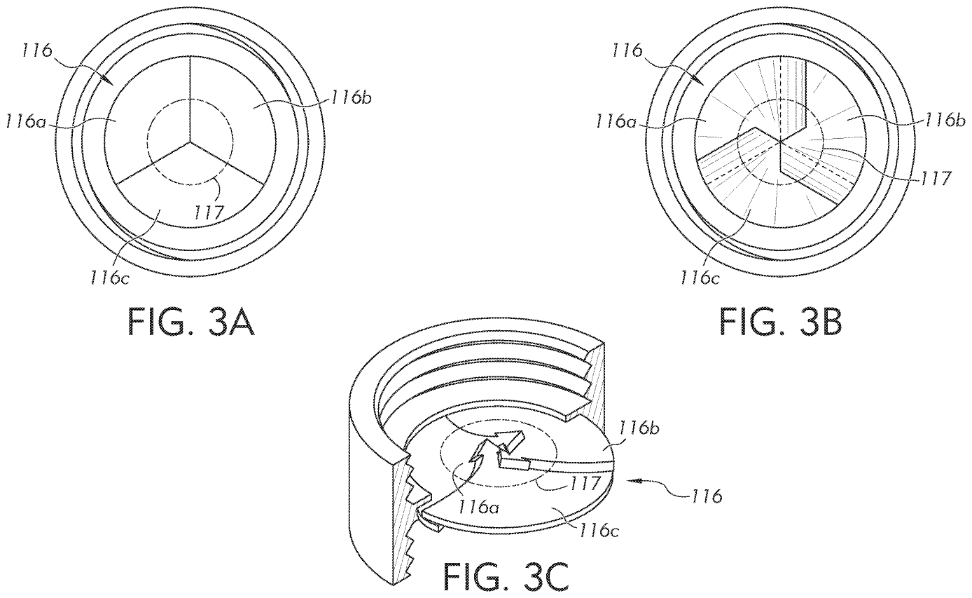

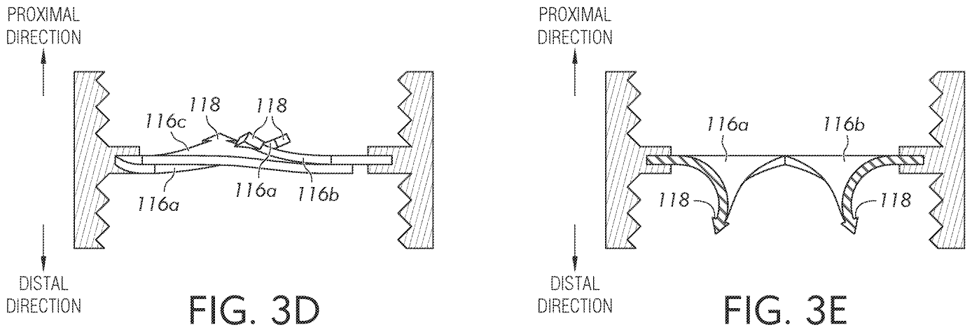

FIGS. 3A-3E illustrate various embodiments of inner seals 116, which may be used in any barrel connector 110 described herein. As depicted in FIG. 3A-3E, the inner seal 116 may include a plurality of flaps 116a-116c. For example, the inner seal may include 2 or more flaps, such as three or more flaps. In the embodiment depicted in FIG. 3A, the inner seal 116 is a tri-flap seal. That is, the inner seal 116 includes three flaps 116a-116c that meet edge to edge in a closed configuration such as depicted. For example, the inner seal 116 may be similar to the HALO Haemostasis valve and/or the LUCOZADE sport bottle cap. When pressure is applied (such as via pressure caused by axially advancing the plunger 102), the inner seal 116 may open to a radius such as denoted by circle 117.

Referring now to FIG. 3B, the inner seal 116 is also illustrated with a plurality of flaps 116a-116c. However, in the present embodiment, adjacent flaps are illustrating as overlapping with one another. Overlapping each flap 116a-116c may assisting in reducing any moisture penetrating the inner seal 116 prior to desired combination. Similar to embodiments above, when pressure is applied (such as by advancing the plunger 102), the flaps 116a-116b may open to a radius denoted by circle 117 allowing the first constituent material 150a to pass through the inner seal 116.

Referring now to FIG. 3C-3E, the inner seal 16 is also illustrated as including a plurality of flaps 116a-116c. It is noted while three flaps are depicted, the inner seal may include any number of flaps. In the present embodiment, the inner seal 116 is a twisting seal. The twisting seal seals the first portion 106a (or first constituent material section 140) from the second portion 106b (or the second constituent material section 142) when in the closed configuration. In the closed configuration, the plurality of flaps 116a-116c twist together to provide a fluid tight seal. Referring specifically to FIG. 3E when pressure is applied (such as by advancing the plunger 102) the plurality of flaps 116a-116c twist apart (e.g., unravel and diverge) to provide an opening between the first portion and the second portion. In embodiments, the plurality of flaps 116a-116c may include engagement edges 118 or hooks such that when the plurality of flaps 116a-116c twist together the engagement edges 118 hook onto the flap around which it twists, such as depicted. In embodiments, the twisting seal may twist in a direction away from the dispensing end 113. That is, the twisted portion of the plurality of flaps may face away from the dispensing end 113 toward the proximal direction in the closed configuration, such as depicted in FIG. 3D. Such may improve sealing against moisture transfer where the liquid component is stored in the first portion 106a and the dry component is stored in the second portion 106b. When actuated to open configuration, such as shown in FIG. 3E, the plurality of flaps extend in the distal direction toward the dispensing end 113.

In each of the embodiments above, the plurality of flaps may be formed of non-fluid penetrable material (e.g., rubber or the like). The plurality of flaps may be formed so as to be biased to the closed configuration. For example, the plurality of flaps may include a shape memory material, such as a shape memory polymer, to hold the plurality of flaps in the closed configuration. In some embodiments, material stiffness selected for the flaps may have a stiffness value greater than the internal pressure being exerted on the flaps (without user input) to allow the plurality of flaps to maintain their position (in a closed state) and then once the user applies additional force via the plunger, the plurality of flaps may fold on themselves and be in an opened state. In some embodiments, the plurality of flaps may be plastically deformed so each flap maintains its closed position. When the user applies force via the plunger, the plurality of flaps may be plastically deformed to push the flaps into the open position. For example, in some embodiments, such as in the twisting seal embodiment, the plurality of flaps may be plastically deformed to point in the opposite direction (e.g., toward the delivery end). In some embodiments, such as in the twisting seal embodiment, the plurality of flaps may be biased to an open configuration. However, due to twisting and overlap, the downward force created may each of the plurality of flaps even out to maintain the plurality of flaps in the closed position until a user (advancing the plunger) generates sufficient force to unravel the twist to allow the plurality of flaps to move to the open position.

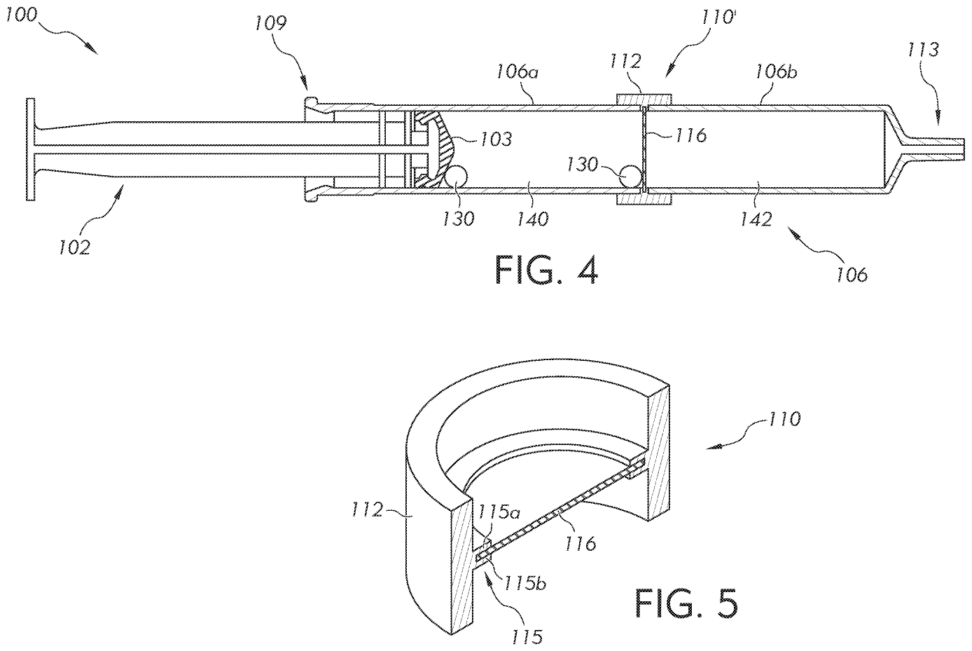

FIG. 4 illustrated the mixing syringe assembly 100 with another embodiment of the barrel connector 110′ and FIG. 5 illustrates the barrel connector 110′ in greater detail. The barrel connector 110′ is substantially the same of as the barrel connector 110 described above accordingly, the above description will not be repeated for brevity. In particular, the barrel connector 110′ includes a barrel body 112 and inner seal 116. However, in the present embodiment there are no threads. Instead, the barrel connector 110′ may be pressfit or overmolded onto the first portion 106a and/or the second portion 106b, thereby coupling the first portion 106a to the second portion 106b.

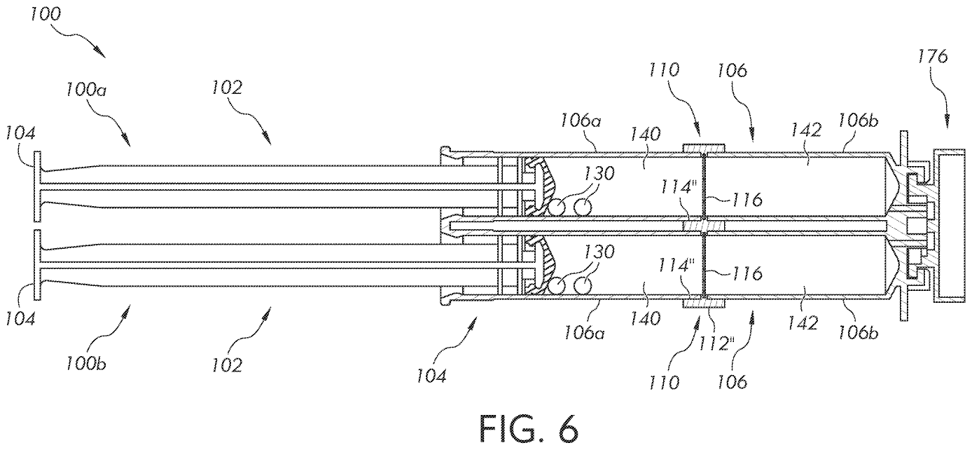

Referring now to FIG. 6, in various embodiments, a mixing syringe assembly 100 may include one or more mixing syringe subassemblies, such as to provide a dual barrel syringe. For example, FIG. 6 illustrates a dual-barrel mixing syringe assembly 100 including a first mixing syringe subassembly 100a and a second mixing syringe subassembly 100b. Each subassembly 100a, 100b is substantially identical to the mixing syringe assembly 100 described with respect to FIG. 1 and/or FIG. 4. In particular, each subassembly 100a may include a plunger 102, a barrel body 106 including a first portion and a second portion, and a barrel connector 110″. The barrel connector 110″ may be similar to either barrel connector 110 or barrel connector 110′, as will be described further below. In some embodiments, the subassemblies 100a, 100b further include one or more mixing masses 130. Accordingly, the above description is applicable to the present embodiments and will not be repeated.

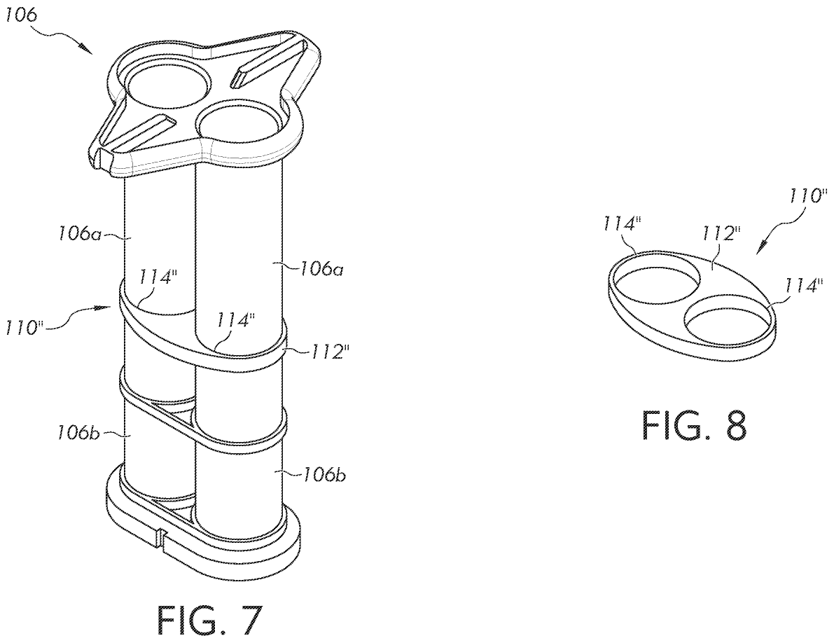

In some embodiments, the respective barrel body portions 106a or 106b may be rigidly coupled. For example, the adjacent second portions 106b of the barrel bodies and/or the adjacent first portion 106a of the barrel bodies 106 may be integrally formed or coupled to another via a bracket, welding, adhesive or the like. Referring to FIG. 7, a dual barrel assembly is depicted without the plungers. In the depicted embodiment, the first portions 106a are rigidly coupled to one another and the second portions 106b are rigidly coupled to one another. The first portions 106a are coupled to the second portions 106b with barrel connector 110″. For example, and as also depicted in FIG. 8, the barrel connector 110″ is a combined barrel connector including a connector body 112″ with two openings 114″ for coupling first portions 106a with corresponding second portions 106b. As in embodiments above, the first and second portions may be pressfit or threaded into the respective openings 114″. In some embodiments, the connector body 112″ may be overmolded over the various first portions and second portions. The barrel connector 110″ may include a seal as described above. It is noted that in embodiments with a threaded connection, the first portions 1106 may not be rigidly coupled to one another prior to threadingly engaging the connector body 112″. Similarly, second portions 106b may not be rigidly coupled to one another prior to threadingly engaging the connector body 112″. It is contemplated that in some embodiments, first or second portions 106a, 106b may be threadingly engaged while the other portion is pressfit or overmolded into the barrel connector 110, 110′, or 110″.

In some embodiments, a manifold 176 may couple the fluid delivery openings of second portions 106b to respective delivery needles or cannulas (not shown) for delivery of fluid. It is noted that in various embodiments, it may be desirable to keep mixed constituent materials from the first subassembly 100a and the second subassembly 100b separate until the point of delivery at a target delivery location, such as within a subject. Accordingly, dual needle assemblies and/or coaxial needle assemblies such as described in International Patent Application No. PCT/US2021/023171, entitled “Multi-Component Sealant Delivery Systems Incorporating Quarter Turn Connectors,” filed Mar. 19, 2021, the entirety of which is hereby incorporated by reference. For example, while the first mixing syringe subassembly 100a may include the first constituent material 150a and the second constituent material 150b, the second mixing syringe subassembly 100b may hold a different first constituent material 150a and/or a different second constituent material 150b. For example, a second constituent material 150b of the first mixing syringe subassembly may hold Peg8, having 20 kmw, while the second constituent material 150b of the second mixing syringe subassembly may hold Peg 8 having 15 kmw. The first constituent materials 150a may also be the same or different and may include, but are not limited to water saline, deionized water, or like. In embodiments, the resulting mixtures or solutions within each of the first and second subassemblies 100a, 100b, may gel or solidify on contact with one another. Accordingly, it may be desirable to only allow contact at the moment of delivery, such as at the end of a dual-lumen cannula, such as disclosed in International Patent Application No. PCT/US2021/023171, entitled “Multi-Component Sealant Delivery Systems Incorporating Quarter Turn Connectors,” filed Mar. 19, 2021, the entirety of which is hereby incorporated by reference.

In use in any of the above embodiments, the plunger 102 is positioned within the first portion 106a initially. When desired, the user may apply a downward force to the plunger 102 causing the diluent to be flushed through the barrel connector 110 (e.g., inner seal) and into the second portion 106b with the powder. Once all the diluent is in the second portion 106b, the plunger 102 may be about halfway down the total length of the barrel body 106, such as at the barrel connector 110. At this stage, the user may hydrate the powder by shaking the assembly 100. Once sufficient hydration of the powder has occurred, the user may then continue to apply downward pressure on the plunger 102, thereby causing the plunger 102 to extend through the barrel connector 110, to deliver the solution through the assembly 100, such as into a delivery needle (not shown). In some embodiments, the material of the seal is highly compressible or very thin to allow passage of the plunger.

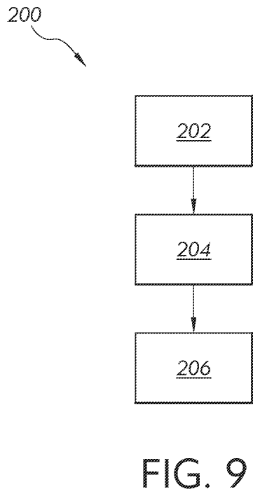

FIG. 9 generally depicts a flow chart illustrating a method 200 of assembling a mixing syringe assembly 100. The method may be used with to construct any of the mixing syringe assemblies described herein. The method 200 may include a greater or fewer number of steps in any order without departing from the scope of the present disclosure. At block 202, the method 200 includes positioning a first constituent material 150a in the first portion 106a of the barrel body 106, such as illustrated in FIG. 1. At block 2024, the method 200 includes positioning the second constituent material 105b in the second portion 106b of the barrel body 106. At block 206, the method 200 includes connecting the first portion 106a of the barrel body 106 to the second portion 106b of the barrel body 106 with a barrel connector 110, as described herein. In embodiments, and as described above, connecting the first portion 106a of the barrel body 106 to the second portion 106b of the barrel body 106 with the barrel connector 110 includes threadingly engaging each of the first portion 106a and the second portion 106b with the barrel connector 110. In some embodiments, connecting the first portion 106a of the barrel body 106 to the second portion 106b of the barrel body 106 with the barrel connector 110 includes engaging each of the first portion 106a and the second portion 106b with the barrel connector 110 via press fit. In some embodiments, connecting the first portion 106a of the barrel body 106 to the second portion 106b of the barrel body 106 with the barrel connector 110 comprises overmolding each of the first portion and the second portion with the barrel connector.

In any of the embodiments described herein, the mixing syringe assembly 100 may be provided to a user as a pre-loaded mixing syringe assembly. That is the mixing syringe assembly 100 may be preloaded with the first constituent material 150a positioned within the first portion and the second constituent material 150b positioned within the second portion. In embodiments wherein the mixing syringe assembly is a dual barrel syringe assembly, each subassembly 100a, 100b may be preloaded with a first constituent material 150a and a second constiuent material 150b, as described above. As in embodiments above, in the closed configuration the inner seal 116 (such as the twisting seal or other disclosed seals), prevents the first constituent material from mixing with the second constituent material and in the open configuration the inner seal 116 the first constituent material is mixable with the second constituent material. That is the first constituent material 150b is able to pass through the inner seal 116 to mix with the second constituent material 150b. Once mixed, the combined substance may be delivered, such as via a needle.

Embodiments may also be described with respect to the following numbered clauses:
1. A mixing syringe assembly for mixing two constituent materials, comprising: a barrel body comprising a first portion and a second portion; and a barrel connector connecting the first portion to the second portion, wherein the barrel connector comprises a twisting seal comprising a plurality of flaps, wherein the twisting seal is operable to be actuated from a closed configuration wherein the twisting seal seals the first portion from the second portion and an open configuration in which the plurality of flaps twist apart to provide an opening between the first portion and the second portion.
2. The mixing syringe assembly of clause 1, wherein the first portion and the second portion have a congruent inner diameter.
3. The mixing syringe assembly of any preceding clause, wherein each of the plurality of flaps include an engagement edge for hooking around an adjacent flap.
4. The mixing syringe assembly of any preceding clause, wherein the barrel connector threadingly engages the first portion and the second portion.
5. The mixing syringe assembly of any of clauses 1-3, wherein the barrel connector is overmolded onto the first portion and the second portion.
6. The mixing syringe assembly of any of clauses 1-3, wherein the first portion is press fit into the barrel connector and the second portion is press fit into the barrel connector.
7. The mixing syringe assembly of any preceding clause, wherein the plurality of flaps comprises 3 or more flaps.
8. The mixing syringe assembly of any preceding clause, wherein the second portion comprises a dispensing end and a twisted portion of the plurality of flaps faces away from the dispensing end of the second portion in the closed configuration.
9. The mixing syringe assembly of clause 8, wherein actuation of the twisting seal causes the plurality of flaps to extend toward the dispensing end of the second portion in the open configuration.
10. The mixing syringe assembly of any preceding clause, wherein the plurality of flaps are biased to the closed configuration.
11. A pre-loaded mixing syringe assembly comprising: a barrel body comprising a first portion and a second portion; a barrel connector connecting the first portion to the second portion, wherein the barrel connector comprises a twisting seal comprising a plurality of flaps, wherein the twisting seal is operable to be actuated from a closed configuration wherein the twisting seal seals the first portion from the second portion and an open configuration in which the plurality of flaps twist apart to provide an opening between the first portion and the second portion; a first constituent material housed in the first portion; and a second constituent material housed in the second portion, wherein in the closed configuration the twisting seal prevents the first constituent material from mixing with the second constituent material and in the open configuration the first constituent material is mixable with the second constituent material.
12. The pre-loaded mixing syringe assembly of clause 11, further comprising a plunger slidably positioned within the barrel body, wherein the plunger is sized to seal to a wall of both the first portion and the second portion.
13. The pre-loaded mixing syringe assembly of clause 12, wherein advancing the plunger along the first portion causes the twisting seal to actuate from the closed configuration to the open configuration.
14. The pre-loaded mixing syringe assembly of any preceding clause, wherein the second portion comprises a dispensing end and a twisted portion of the plurality of flaps faces away from the dispensing end of the second portion in the closed configuration.
15. The pre-loaded mixing syringe assembly of clause 14, wherein actuation of the twisting seal causes the plurality of flaps to extend toward the dispensing end of the second portion in the open configuration.
16. The pre-loaded mixing syringe assembly of any preceding clause, wherein the plurality of flaps are biased to the closed configuration.
17. A method of assembling a mixing syringe assembly, the method comprising: positioning a first constituent material in a first portion of a barrel body; positioning a second constituent in a second portion of the barrel body; and connecting the first portion of the barrel body to the second portion of the barrel body with a barrel connector, the barrel connector comprising a twisting seal comprising a plurality of flaps, wherein the twisting seal is operable to be actuated from a closed configuration wherein the twisting seal seals the first portion from the second portion and an open configuration in which the plurality of flaps twist apart to provide an opening between the first portion and the second portion.
18. The method of clause 17, wherein connecting the first portion of the barrel body to the second portion of the barrel body with the barrel connector comprises threadingly engaging each of the first portion and the second portion with the barrel connector.
19. The method of clause 17, wherein connecting the first portion of the barrel body to the second portion of the barrel body with the barrel connector comprises engaging each of the first portion and the second portion with the barrel connector via press fit.
20. The method of clause 17, wherein connecting the first portion of the barrel body to the second portion of the barrel body with the barrel connector comprises overmolding each of the first portion and the second portion with the barrel connector.

It should now be understood that the present disclosure relates to various mixing syringe assemblies and methods of mixing constituent materials with mixing syringe assemblies. The various embodiments provided herein may provide ready to use or easily assembled syringe assemblies for easily mixing components. Moreover, embodiments as provided herein may assist in maintaining sterility and/or constituent material integrity, while improving ease of mixing and delivery.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A mixing syringe assembly for mixing two constituent materials, comprising:
a barrel body comprising a first portion and a second portion; and
a barrel connector connecting the first portion to the second portion, wherein the barrel connector comprises a twisting seal comprising a plurality of flaps, wherein the twisting seal is operable to be actuated from a closed configuration wherein the twisting seal seals the first portion from the second portion and an open configuration in which the plurality of flaps twist apart to provide an opening between the first portion and the second portion.

2. The mixing syringe assembly of claim 1, wherein the first portion and the second portion have a congruent inner diameter.

3. The mixing syringe assembly of claim 1, wherein each of the plurality of flaps include an engagement edge for hooking around an adjacent flap.

4. The mixing syringe assembly of claim 1, wherein the barrel connector threadingly engages the first portion and the second portion.

5. The mixing syringe assembly of claim 1, wherein the barrel connector is overmolded on the first portion and the second portion.

6. The mixing syringe assembly of claim 1, wherein the first portion is press fit into the barrel connector and the second portion is press fit into the barrel connector.

7. The mixing syringe assembly of claim 1, wherein the plurality of flaps comprises 3 or more flaps.

8. The mixing syringe assembly of claim 1, wherein the second portion comprises a dispensing end and a twisted portion of the plurality of flaps faces away from the dispensing end of the second portion in the closed configuration.

9. The mixing syringe assembly of claim 8, wherein actuation of the twisting seal causes the plurality of flaps to extend toward the dispensing end of the second portion in the open configuration.

10. The mixing syringe assembly of claim 1, wherein the plurality of flaps are biased to the closed configuration.

11. A pre-loaded mixing syringe assembly comprising:
a barrel body comprising a first portion and a second portion;
a barrel connector connecting the first portion to the second portion, wherein the barrel connector comprises a twisting seal comprising a plurality of flaps, wherein the twisting seal is operable to be actuated from a closed configuration wherein the twisting seal seals the first portion from the second portion and an open configuration in which the plurality of flaps twist apart to provide an opening between the first portion and the second portion;
a first constituent material housed in the first portion; and
a second constituent material housed in the second portion, wherein in the closed configuration the twisting seal prevents the first constituent material from mixing with the second constituent material and in the open configuration the first constituent material is mixable with the second constituent material.

12. The pre-loaded mixing syringe assembly of claim 11, further comprising a plunger slidably positioned within the barrel body, wherein the plunger is sized to seal to a wall of both the first portion and the second portion.

13. The pre-loaded mixing syringe assembly of claim 12, wherein advancing the plunger along the first portion causes the twisting seal to actuate from the closed configuration to the open configuration.

14. The pre-loaded mixing syringe assembly of claim 11, wherein the second portion comprises a dispensing end and a twisted portion of the plurality of flaps faces away from the dispensing end of the second portion in the closed configuration.

15. The pre-loaded mixing syringe assembly of claim 14, wherein actuation of the twisting seal causes the plurality of flaps to extend toward the dispensing end of the second portion in the open configuration.

16. The pre-loaded mixing syringe assembly of claim 11, wherein the plurality of flaps are biased to the closed configuration.

17. A method of assembling a mixing syringe assembly, the method comprising:
positioning a first constituent material in a first portion of a barrel body;
positioning a second constituent in a second portion of the barrel body; and
connecting the first portion of the barrel body to the second portion of the barrel body with a barrel connector, the barrel connector comprising a twisting seal comprising a plurality of flaps, wherein the twisting seal is operable to be actuated from a closed configuration wherein the twisting seal seals the first portion from the second portion and an open configuration in which the plurality of flaps twist apart to provide an opening between the first portion and the second portion.

18. The method of claim 17, wherein connecting the first portion of the barrel body to the second portion of the barrel body with the barrel connector comprises threadingly engaging each of the first portion and the second portion with the barrel connector.

19. The method of claim 17, wherein connecting the first portion of the barrel body to the second portion of the barrel body with the barrel connector comprises engaging each of the first portion and the second portion with the barrel connector via press fit.

20. The method of claim 17, wherein connecting the first portion of the barrel body to the second portion of the barrel body with the barrel connector comprises overmolding each of the first portion and the second portion with the barrel connector.
